(19) United States
(12) Patent Application Publication
(10) Pub. No.: US 2024/0358317 A1
(43) Pub. Date: Oct. 31, 2024

(54) SLEEP DETECTION WITH MULTIPLE SENSORS

(71) Applicant: Samsung Electronics Co., Ltd., Suwon-si (KR)

(72) Inventors: Lianjun Li, McKinney, TX (US); Hao Chen, Allen, TX (US); Hao-Hsuan Chang, Plano, TX (US); Se Ho Kim, Richardson, TX (US); Doyoon Kim, Suwon (KR); Joonhyun Lee, Seoul (KR)

(21) Appl. No.: 18/522,138

(22) Filed: Nov. 28, 2023

Related U.S. Application Data
(60) Provisional application No. 63/461,868, filed on Apr. 25, 2023.

(51) Int. Cl. A61B 5/00

(52) U.S. Cl. CPC A61B 5/4812; A61B 5/7225; A61B 5/7246; A61B 5/7271

(57) ABSTRACT

A sleep monitoring apparatus includes a plurality of sensor modules, a transceiver, and a processor operatively coupled with the plurality of sensor modules and the transceiver. The processor is configured to receive, from the plurality of sensor modules, raw sensor data for each of the plurality of sensor modules related to a sleep session of a user of the sleep monitoring apparatus, and perform raw data fusion of the raw sensor data. The raw data fusion generates a fused raw data signal. The processor is further configured to, based on the fused raw data signal, perform feature extraction for the plurality of sensor module; based on the feature extraction, perform feature fusion for the plurality of sensor modules; based on the feature fusion, perform decision fusion for the plurality of sensor modules; and determine a sleep stage of the user based on the decision fusion.

### CROSS-REFERENCE TO RELATED APPLICATION AND CLAIM OF PRIORITY

This application claims priority under 35 U.S.C. § 119 (e) to U.S. Provisional Patent Application No. 63/461,868 filed on Apr. 25, 2023. The above-identified provisional patent application is hereby incorporated by reference in its entirety.

### TECHNICAL FIELD

This disclosure relates generally to consumer devices. More specifically, this disclosure relates to methods and apparatuses for sleep detection with multiple sensors.

### BACKGROUND

Sleep stage detection is a very challenging problem. Most of the current solutions for detecting a sleep stage focus on just one sensor, for example an ultrawide band (UWB) sensor or a microphone. The accuracy typically for such solutions is very low. For example, the typical sleep stage detection accuracy is around 70%. Polysomnography (PSG) is the typical way to capture accurate sleep stage data. In PSG the most important parameter is the brain wave from an Electroencephalogram (EEG). However, it is difficult to embed EEG sensors in existing commercial devices. Therefore, an improved sleep stage detection solution is desirable.

### SUMMARY

This disclosure provides methods and apparatuses for sleep detection with multiple sensors.

In one embodiment, a sleep monitoring apparatus is provided. The sleep monitoring apparatus includes a plurality of sensor modules, a transceiver, and a processor operatively coupled with the plurality of sensor modules and the transceiver. The processor is configured to receive, from the plurality of sensor modules, raw sensor data for each of the plurality of sensor modules related to a sleep session of a user of the sleep monitoring apparatus, and perform raw data fusion of the raw sensor data. The raw data fusion generates a fused raw data signal. The processor is further configured to, based on the fused raw data signal, perform feature extraction for the plurality of sensor module; based on the feature extraction, perform feature fusion for the plurality of sensor modules; based on the feature fusion, perform decision fusion for the plurality of sensor modules; and determine a sleep stage of the user based on the decision fusion.

In another embodiment, method of operating a sleep monitoring apparatus is provided. The method includes receiving, from a plurality of sensor modules, raw sensor data for each of the plurality of sensor modules related to a sleep session of a user of the sleep monitoring apparatus, and performing raw data fusion of the raw sensor data. The raw data fusion generates a fused raw data signal. The method further includes, based on the fused raw data signal, performing feature extraction for the plurality of sensor modules; based on the feature extraction, performing feature fusion for the plurality of sensor modules; based on the feature fusion, performing decision fusion for the plurality of sensor modules; and determining a sleep stage of the user based on the decision fusion.

In yet another embodiment, a non-transitory computer readable medium embodying a computer program is provided. The computer program includes program code that, when executed by a processor of a device, causes the device to receive, from a plurality of sensor modules, raw sensor data for each of the plurality of sensor modules related to a sleep session of a user of the sleep monitoring apparatus, and perform raw data fusion of the raw sensor data. The raw data fusion generates a fused raw data signal. The computer program further includes program code that, when executed by a processor of a device, causes the device to, based on the fused raw data signal, perform feature extraction for the plurality of sensor modules; based on the feature extraction, perform feature fusion for the plurality of sensor modules; based on the feature fusion, perform decision fusion for the plurality of sensor modules; and determine a sleep stage of the user based on the decision fusion.

Other technical features may be readily apparent to one skilled in the art from the following figures, descriptions, and claims.

Before undertaking the DETAILED DESCRIPTION below, it may be advantageous to set forth definitions of certain words and phrases used throughout this patent document. The term “couple” and its derivatives refer to any direct or indirect communication between two or more elements, whether or not those elements are in physical contact with one another. The terms “transmit,” “receive,” and “communicate,” as well as derivatives thereof, encompass both direct and indirect communication. The terms “include” and “comprise,” as well as derivatives thereof, mean inclusion without limitation. The term “or” is inclusive, meaning and/or. The phrase “associated with,” as well as derivatives thereof, means to include, be included within, interconnect with, contain, be contained within, connect to or with, couple to or with, be communicable with, cooperate with, interleave, juxtapose, be proximate to, be bound to or with, have, have a property of, have a relationship to or with, or the like. The term “controller” means any device, system or part thereof that controls at least one operation. Such a controller may be implemented in hardware or a combination of hardware and software and/or firmware. The functionality associated with any particular controller may be centralized or distributed, whether locally or remotely. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

Definitions for other certain words and phrases are provided throughout this patent document. Those of ordinary skill in the art should understand that in many if not most instances, such definitions apply to prior as well as future uses of such defined words and phrases.

### BRIEF DESCRIPTION OF THE DRAWINGS

For a more complete understanding of this disclosure and its advantages, reference is now made to the following description, taken in conjunction with the accompanying drawings, in which:

FIG. 1 illustrates an example of hardware components for a smart sleep solution divided into several main categories according to embodiments of the present disclosure;

FIG. 2 illustrates an example of software components for a smart sleep solution according to embodiments of the present disclosure;

FIG. 3 illustrates an example electronic device according to embodiments of the present disclosure;

FIG. 4 illustrates a block diagram for an example smart chair system and sensor fusion components according to embodiments of the present disclosure;

FIG. 5 illustrates an example sensor fusion architecture according to embodiments of the present disclosure;

FIG. 6 illustrates a block diagram for an example of combined sensor fusion according to embodiments of the present disclosure;

FIGS. 7A-7B illustrate example solutions for raw data level fusion according to embodiments of the present disclosure;

FIGS. 8A-8C illustrate example solutions for feature level fusion according to embodiments of the present disclosure;

FIGS. 9A-9F illustrate example solutions for decision level fusion according to embodiments of the present disclosure; and

FIG. 10 illustrates a method for sleep detection with multiple sensors according to embodiments of the present disclosure.

### DETAILED DESCRIPTION

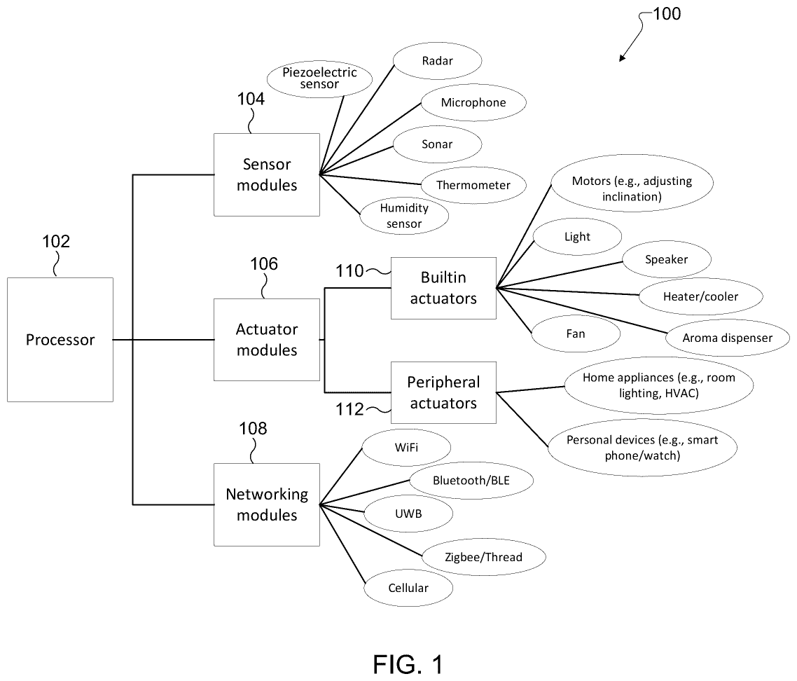

FIGS. 1 through 10, discussed below, and the various embodiments used to describe the principles of this disclosure in this patent document are by way of illustration only and should not be construed in any way to limit the scope of the disclosure. Those skilled in the art will understand that the principles of this disclosure may be implemented in any suitably arranged smart sleep solution.

In this disclosure, leveraging various non-invasive sensors (e.g., not restricting user's body movement and/or causing any discomfort), various methods and apparatuses are described to detect the sleep stage of a user as well as those events affecting the sleep quality. Such apparatuses may be referred to as a sleep monitoring apparatus, a smart sleep solution, a smart sleep chair, etc. Additionally, per the detection and monitoring of the sleep stages and relevant events, methods and apparatus to take certain actions to improve the user's sleep quality are disclosed. In this disclosure, a device that implements these capabilities may be referred to as a smart sleep solution. However, it should be understood that the disclosed methods and apparatuses be implemented in different forms such as a chair, a bed, a couch or similar furniture. Such forms may be referred to as a smart sleep chair, a smart chair, a smart bed, a smart couch, etc. Furthermore, it should be understood that the disclosed methods and apparatuses may also be implemented in different forms that allow the disclosed methods and apparatuses to be utilized as a peripheral device for standalone furniture, such as a chair, a bed, a couch, or similar.

A smart sleep solution as described herein may comprise two types of hardware components to implement the functionality of this disclosure:
1. Various non-invasive sensors for sleep stage and relevant events detection and monitoring: Some examples of these sensors include radar (at various radio frequencies),
2. Various control devices that can take certain actions to improve sleep quality: Some examples include actuators that can adjust the inclination angle of a chair, speakers (e.g., playing some soothing music), electronics/appliance controllers (e.g., lighting, AC in the room etc.), wireless network capability that enables connection to peripherals (e.g., personal devices) to control and/or adjust some settings, etc.

FIG. 1 illustrates an example of hardware components 100 for a smart sleep solution divided into several main categories according to embodiments of the present disclosure. The embodiment of hardware components of FIG. 1 is for illustration only. Different embodiments of hardware component could be used without departing from the scope of this disclosure.

In the example of FIG. 1, the smart sleep solution has a main processor 102 that is connected to all other modules. It processes input from those modules, makes determinations of whether certain actions should be taken (e.g., aiming to improve sleep quality), and if actions are needed, it may output control signals to the appropriate entities. The components under the processor control may fall into one of three main categories:
1. Sensor modules 104: These are sensor devices that can be used to monitor the sleep stages and detect related events (e.g., limb/body movement, snoring, etc.). Examples of such sensors include radar, sonar, thermometer, humidity sensor, microphone, piezoelectric sensor, etc.
2. Actuator modules 106: These are devices that could influence the environment and can affect the user's sleep. One aim of a smart sleep solution is to determine proper actions allowable by these actuator devices to improve the user's sleep quality. Actuator modules may be further divided into two types: built-in and peripheral.
   a. Built-in actuators 110: These are devices equipped on the smart sleep solution itself. Some examples include motors (e.g., for adjusting the reclining of a chair or bed), speaker, lights, heater/cooler (e.g., something like an electric blanket or similar function could be embedded in a chair's cushion), fan, etc.
   b. Peripheral actuators 112: These are actuator devices in the vicinity of the smart sleep solution (e.g., inside the room) that could be connected to the smart sleep solution by the networking module. For example, these may include home appliances such as HVAC appliances, room lighting, air purifier, fan, etc. Another set of examples includes personal devices such as a smart phone or a smart watch (e.g., with the user's permission, the smart sleep solution could change the setting to avoid disturbances from those devices).
3. Networking modules 108: These provide connectivity capability for the smart sleep solution. For example, the smart sleep solution may use networking modules to connect to the peripheral actuators. Examples of network modules include transceivers for WiFi, Bluetooth/BLE, Ultrawideband (UWB), Zigbee/Thread, cellular such as LTE/5G, etc. In some embodiments, networking modules may be used to connect the smart sleep solution to a remote apparatus, such as a cloud server on the internet, etc.

Although FIG. 1 illustrates one example of hardware components 100 for a smart sleep solution, various changes may be made to FIG. 1. For example, the smart sleep solution could include any number of each component shown in FIG. 1. Also, various components in FIG. 1 could be combined, further subdivided, or omitted and additional components could be added according to particular needs.

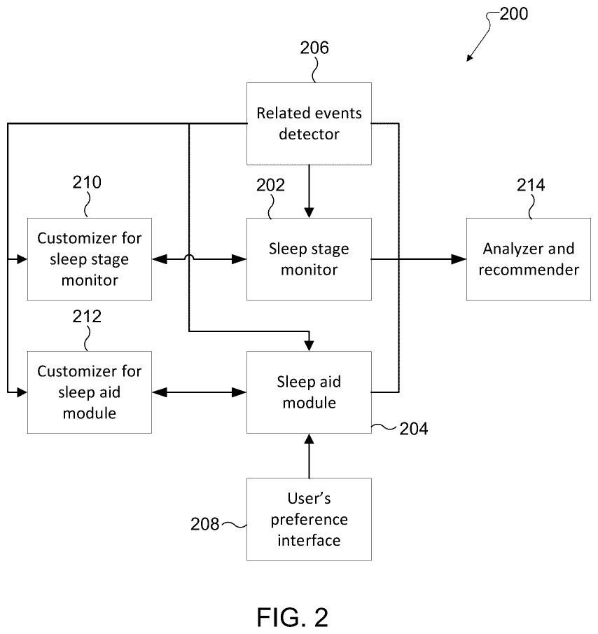

A primary purpose of a smart sleep solution as described herein is to monitor and improve the user's sleep quality. Software needed to support this goal may be divided into several components as illustrated in FIG. 2.

FIG. 2 illustrates an example 200 of software components for a smart sleep solution according to embodiments of the present disclosure. The embodiment of software components of FIG. 2 is for illustration only. Different embodiments of software components could be used without departing from the scope of this disclosure.

The core functionality of a smart sleep solution as described herein may lie in the sleep stage monitoring and the sleep aid module. The sleep aid module may allow the user to input their preference setting. The related events detector could be used to detect those events that may have impact on the sleep quality such as snoring, sleep apnea, etc. The customizers personalize the solutions (the sleep stage monitoring and sleep aid module) aiming at improving performance over time and adapt to the user's preferences and habits. Finally, the history from the sleep stage monitoring, the sleep aid module, and related events detector may be processed by the analyzer, which could provide some summary and related sleep quality metrics to the user. Further, those inputs may also be used to make recommendations to suggest some actions to the user that they may take to improve their sleep quality. Some details of the main functionalities of each component are provided below:
Sleep stage monitor (202): This module uses the sensing information from the sensor modules of the hardware component to detect and monitor the sleep state and sleep stage of the user. Sleep state may refer to, for example, the user being awake and attempting to sleep, actively sleeping, sleeping during a period where the user should be awakened, etc. Sleep stage may refer to the four sleep stages N1, N2, N3, and Rapid Eye Movement (REM). The sensing information may be processed into some intermediate format. For example, radar measurements may be processed to estimate the vital sign (e.g., breathing rate, heart rate, etc.) as well as other notable body movements.
Sleep aid module (204): This component uses the detected sleep states, sleep stages, related events, as well as user preference to take certain actions (e.g., by activating one or more of the actuators belonging to the actuator module) to facilitate and/or enhance the user's sleep quality. Note that the sleep aid module can operate at different states of the sleep including the duration for the user to fall asleep, when the user is asleep, as well as during the process of waking up.
Related events detector (206): This module is responsible for detecting various events that could affect the user's sleep quality. It uses the sensing information from the sensing module and may or may not use the same set of sensing information as used by the sleep stage monitoring module. Some examples of related events include snoring, limb/body movement, teeth grinding, night terror, sleep apnea, etc.
User's preference interface (208): As sleep habits vary a great deal for different individuals, different preferences for different users can be expected. For example, certain users may prefer to have light soothing music in the background to help them fall asleep, while other users may prefer complete silence. Similarly, some users may benefit from a preferred aromatic scent. These kinds of preferences that relate to ease of falling asleep and/or maintaining sleep quality could be provided through this interface.
Customizer for sleep stage monitor (210): Because of the large variation across users, it can be expected that there exists room for improvement for a generic solution (that aims at all or a large group of users). Such a generic solution is designed to work well for all targeted users, but may not be the best for any given user. Therefore, customization to tune and maximize the performance for the user could be beneficial. The purpose of this module is to collect related sensing information at times when it is determined that the detected sleep stage from the current solution might be incorrect. This is based on the fact that there are certain events that only occur in a specific sleep stage. Thus, if such an event is detected and the detected sleep stage is not consistent (i.e., that event could not occur in this sleep stage), then it is determined to be a likely wrong detection and this module would log the sensing information along with the expected sleep stage as the label. Such data could be used as additional training data personalized to the user, which could be used to fine-tune the classifier model to improve the performance.
Customizer for sleep aid module (212): The purpose of this module is similar to the customizer for sleep stage detector module, but with the focus on the sleep aid. Certain users may respond better to certain actions than others, and thus a one-size-fits-all solution may likely underperform. For example, in adjusting a chair reclining angle in response to a snoring event, the optimal angle would likely depend on the physique of the user as well as their personal preference. This module may request feedback from the user such as asking the user to rate the quality of the sleep session or some other related aspects. It may also use the sensing information (i.e., implicit feedback) to do the customization as well.
Analyzer and recommender (214): The detected sleep stages, related events, as well as responses from sleep aid module could be used to analyze the overall sleep quality. Such information may also be used to make certain recommendations for the user, for example, by providing some suggestions for adjusting their lifestyle that could help improve their sleep quality. Another aspect is that there are events that the smart sleep solution cannot respond to. E.g., if teeth grinding is detected, the recommender may suggest the user to consult a dentist and/or use a night guard.

Although FIG. 2 illustrates one example 200 of software components for a smart sleep solution, various changes may be made to FIG. 2. For example, the smart sleep solution could include any number of each component shown in FIG. 2. Also, various components in FIG. 2 could be combined, further subdivided, or omitted and additional components could be added according to particular needs.

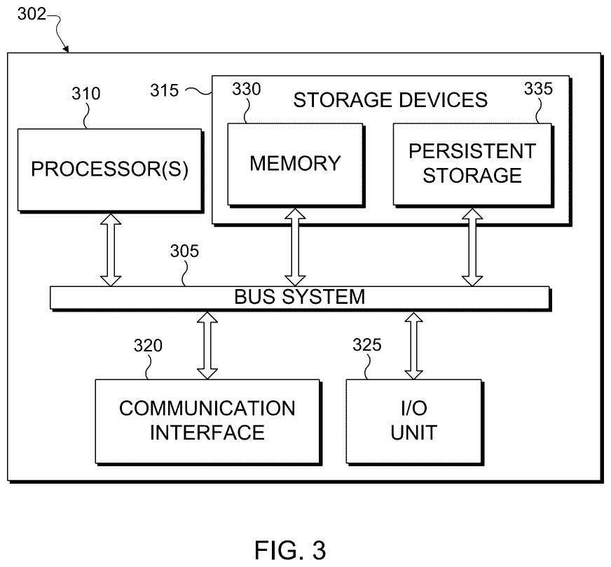

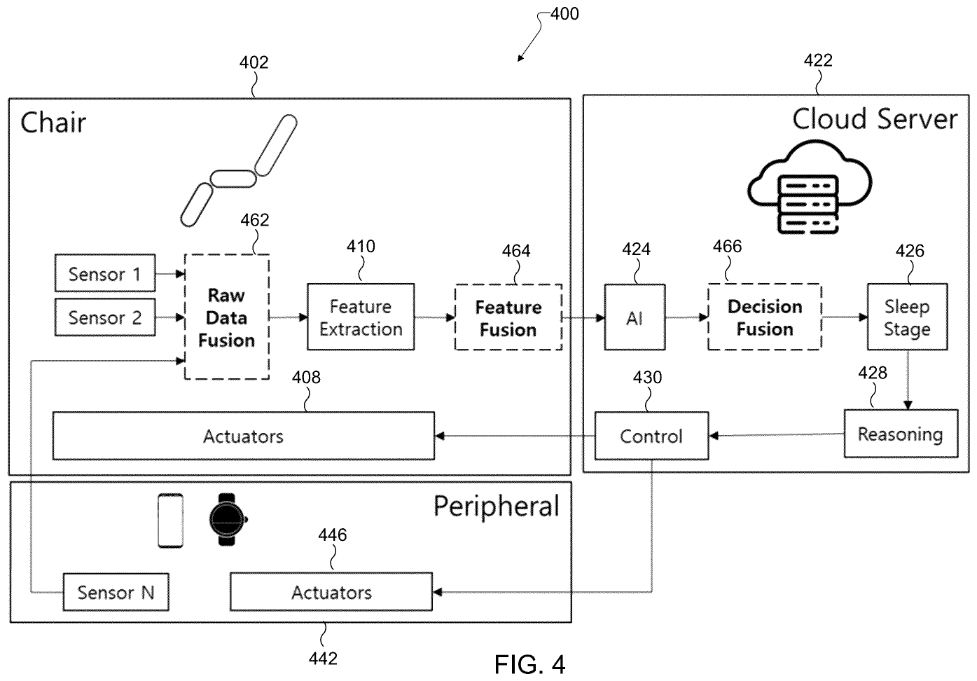

FIG. 3 illustrates an example electronic device according to embodiments of the present disclosure. In particular, FIG. 3 illustrates an example server 302 that may be operatively coupled to smart chair 402 of FIG. 4, and the server 302 could represent the processor 102 in FIG. 1. The server 302 can represent one or more processors, local servers, remote servers, clustered computers, and components that act as a single pool of seamless resources, a cloud-based server, and the like. The server 302 can be accessed by one or more of processor 102 and modules 104-108 of FIG. 1 or another server.

As shown in FIG. 3, the server 302 includes a bus system 305 that supports communication between at least one processing device (such as a processor 310), at least one storage device 315, at least one communications interface 320, and at least one input/output (I/O) unit 325. The server 302 can represent one or more local servers, one or more remote servers, can be integrated directly into another apparatus such as smart sleep solution in FIG. 2, or can be communicatively coupled with another apparatus such as smart sleep solution in FIG. 2.

The processor 310 executes instructions that can be stored in a memory 330. The processor 310 can include any suitable number(s) and type(s) of processors or other devices in any suitable arrangement. Example types of processors 310 include microprocessors, microcontrollers, digital signal processors, field programmable gate arrays, application specific integrated circuits, and discrete circuitry.

The memory 330 and a persistent storage 335 are examples of storage devices 315 that represent any structure(s) capable of storing and facilitating retrieval of information (such as data, program code, or other suitable information on a temporary or permanent basis). The memory 330 can represent a random-access memory or any other suitable volatile or non-volatile storage device(s). For example, the instructions stored in the memory 330 can include instructions for enhancing sleep quality. The persistent storage 335 can contain one or more components or devices supporting longer-term storage of data, such as a read only memory, hard drive, flash memory, or optical disc.

The communications interface 320 supports communications with other systems or devices. For example, the communications interface 320 could include a network interface card or a wireless transceiver facilitating communications with networking modules 108 of FIG. 1. The communications interface 320 can support communications through any suitable physical or wireless communication link(s). For example, the communications interface 320 can transmit a bitstream containing user information another device such as smart sleep solution in FIG. 2.

The I/O unit 325 allows for input and output of data. For example, the I/O unit 325 can provide a connection for user input through a keyboard, mouse, keypad, touchscreen, or other suitable input device. The I/O unit 325 can also send output to a display, printer, or other suitable output device. Note, however, that the I/O unit 325 can be omitted, such as when I/O interactions with the server 302 occur via a network connection.

Note that while FIG. 3 may be described as representing the processor 102 of FIG. 1, the same or similar structure could be used in other devices or elements, including one or more of cloud server 422, and peripheral 442 of FIG. 4 For example, cloud server 422 and peripheral 442 could have the same or similar structure as that shown in FIG. 3.

Sleep has a critical impact on health. Sufficiently long and good sleep quality is critical for waking up feeling refreshed and be energetic during the day. Yet, in modern societies across the globe, sleep deprivation is a common problem. For example, according to some studies, almost half of US adults report feeling sleepy between three to seven days per week. While the recommended sleep duration for adults (16-64) is 7-8 hours, 35.2% of all US adults report average sleep duration of less than seven hours. This is a common trend observed all over the world. Apart from the sleep duration, the sleep quality is also very important.

Sleep is a rather complicated biological process, and it is not static but rather a dynamic process. One typical night of sleep for an adult can include four to six rounds of sleep cycle, which is composed of four sleep stages. The sleep cycles are not uniform, but on average a sleep cycle lasts about 90 minutes.

For good quality of sleep, it is critical that the body progresses smoothly through those sleep cycles. Furthermore, certain events, such as sleep apnea, can also affect the sleep quality. In this disclosure, leveraging various non-invasive sensors (e.g., not restricting user's body movement and/or causing any discomfort), sleep stage as well as events affecting sleep quality can be detected (or identified, determined, etc.).

In the present disclosure, methods and apparatus to detect and monitor sleep stage and relevant body events with multiple non-invasive sensors are described. These sensors may include ultrasound wide band (UWB), millimeter wave (mmWave) radar, ultrasound, piezo-electric sensor, audio etc. At a high level, the solution has one set of sensors and a set of algorithms to enable the disclosed methods and apparatus. The two components are:
1. Various non-invasive sensors for sleep stage and relevant events detection and monitoring: Some examples of these sensors include radar (at various radio frequencies),
2. Various algorithms to process the data from these radars and especially the sensor fusion algorithm to output the sleep stage from these radar signals.

FIG. 4 illustrates a block diagram for an example smart chair system and sensor fusion components 400 according to embodiments of the present disclosure. The embodiment of a smart chair system and sensor fusion components 400 of FIG. 4 is for illustration only. Different embodiments of a smart chair system and sensor fusion components 400 could be used without departing from the scope of this disclosure.

In the example of FIG. 4, a smart sleep solution in the form of a smart chair system and sensor fusion components are illustrated. The smart chair system includes smart chair 402, cloud server 422 and peripheral 442. Smart chair 402, cloud server 422, and peripheral 442 may be similar as described regarding FIGS. 1-3.

In the example of FIG. 4, smart chair 402 further includes sensors 1 and 2, and actuators 408, and peripheral 442 further includes sensor N and actuators 446. While peripheral 442 is illustrated as a watch and/or a wireless telephone, it should be understood that peripheral 442 is not limited to any particular form factor or hardware implementation. Likewise, while smart chair 402 is illustrated as a chair, it should be understood that smart chair 402 is not limited to any particular form factor or hardware implementation of a smart sleep solution. Similarly, while cloud server 422 is depicted as a remote cloud server it should be understood that cloud server 422 is not limited to any particular implementation. For example, cloud server 422 could also be a local server, integrated into smart chair 402, integrated into peripheral 442, etc.

Where multi-modality sensors (e.g., mmWave, UWB, Piezo) either placed in smart chair 402 (e.g., sensor 1 and/or sensor 2) or worn by the user (e.g., sensor N) are utilized to collect raw sensing signals. Those signals are then utilized to extract vital sign related features at block 410. The extraction of vital sign related features may be referred to as feature extraction. Finally, sleep stages are detected by AI model 424 based on those features at block 426. Given a particular sleep stage, different actions can be conducted by reasoning block 428 at control block 430 to improve the user's sleep experience. Within this pipeline, sensor fusion could happen in three places, as marked in the dotted blocks, which are raw data fusion 462, feature fusion 464, and decision fusion 466.

Although FIG. 4 illustrates a block diagram for an example smart chair system and sensor fusion components 400, various changes may be made to FIG. 4. For example, the smart chair system and sensor fusion components 400 could include any number of each component described with respect to FIG. 4. Also, various components described with respect to FIG. 4 could be combined, further subdivided, located in alternative locations, or omitted and additional components could be added according to particular needs.

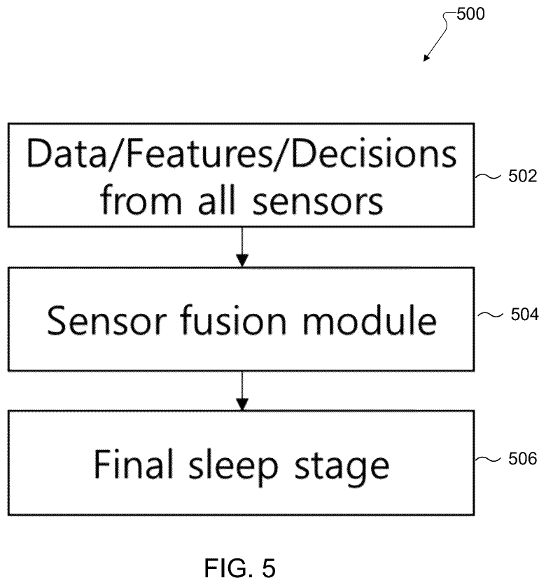

FIG. 5 illustrates an example sensor fusion architecture 500 according to embodiments of the present disclosure. The example sensor fusion architecture 500 of FIG. 5 is for illustration only. Different embodiments of sensor fusion architecture 500 could be used without departing from the scope of this disclosure.

In the example of FIG. 5, The sensor fusion module 504 will take each sensor data or features or decision 502 and then make the final decision of the sleep stage 506. In one of the embodiments, the sensor fusion algorithm will take the raw data from each sensor, and output the sleep stage. In another embodiment, the sensor fusion module will take features from each sensor and output the sleep stage. In another embodiment, the sensor fusion module will take soft decision on sleep stage from each sensor and output the final sleep stage. In yet another embodiment, the sensor fusion module will take hard decision results on sleep stage from each sensor and then output the final sleep stage decision results.

Although FIG. 5 illustrates one example of a sensor fusion architecture 500, various changes may be made to FIG. 5. For example, while shown as a series of steps, various steps in FIG. 5 could overlap, occur in parallel, occur in a different order, or occur any number of times.

In the present disclosure, different levels of sensor fusion algorithms are described. In one embodiment, raw data from each sensor is fused to improve the signal to noise ratio (SNR) of the sensor data. This level of fusion may be referred to as raw data level fusion.

In another embodiment, feature level fusion is described where some features (for example processed spectrogram or vital signs) can be fused so that the feature processed will be more accurate.

In yet another embodiment, decision level fusion is described where each sensor could output the decision of sleep stage. For decision level fusion, the fusion model may take AI output and make a final decision on sleep stage. In decision level fusion, one method, which may be referred to as soft fusion, is described where the probability of sleep stage is fused. For example, each sensor may output a probability of Wake/Light/REM/Deep, and the probability from each sensor could be processed or averaged to get the final sleep stage. Another method, which may be referred to as hard fusion, is described where the hard decision of each sleep stage is fused. For example, each sensor may output Wake/Light/REM/Deep, and sensor fusion may take these outputs to run algorithms.

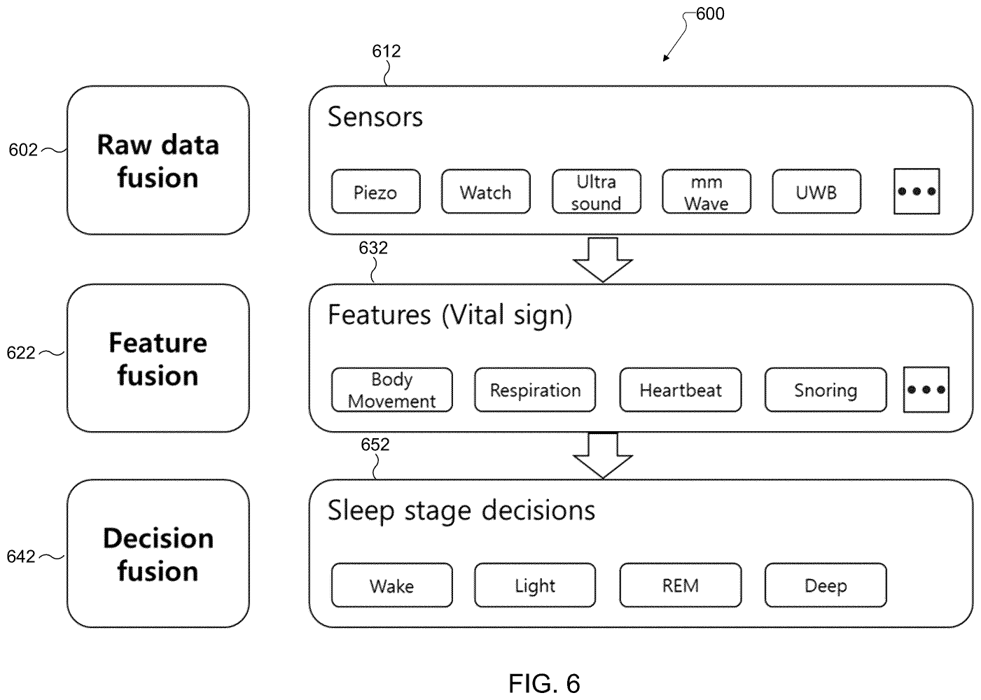

In yet another embodiment, all these different levels of fusion are combined as shown in FIG. 6.

FIG. 6 illustrates a block diagram for an example of combined sensor fusion 600 according to embodiments of the present disclosure. The embodiment of combined sensor fusion 600 of FIG. 6 is for illustration only. Different embodiments of combined sensor fusion 600 could be used without departing from the scope of this disclosure.

In the example of FIG. 6, raw data level fusion 602, feature level fusion 622, and decision level fusion 642 are combined. For example, raw data from sensors 612 are fused at raw data level fusion 602. The fused data from raw data level fusion 602 flows into feature level fusion 622, and features 632 (e.g., vital signs) are fused at feature level fusion 622. Finally the fused data from feature level fusion 622 flows into decision level fusion 642, and decisions 652 (e.g. sleep stage decisions) are fused at decision level fusion 642.

Although FIG. 6 illustrates a block diagram for an example of combined sensor fusion 600, various changes may be made to FIG. 6. For example, the implementation of combined sensor fusion 600 could include any number of each component described with respect to FIG. 6. Also, various components described with respect to FIG. 6 could be combined, further subdivided, located in alternative locations, or omitted and additional components could be added according to particular needs.

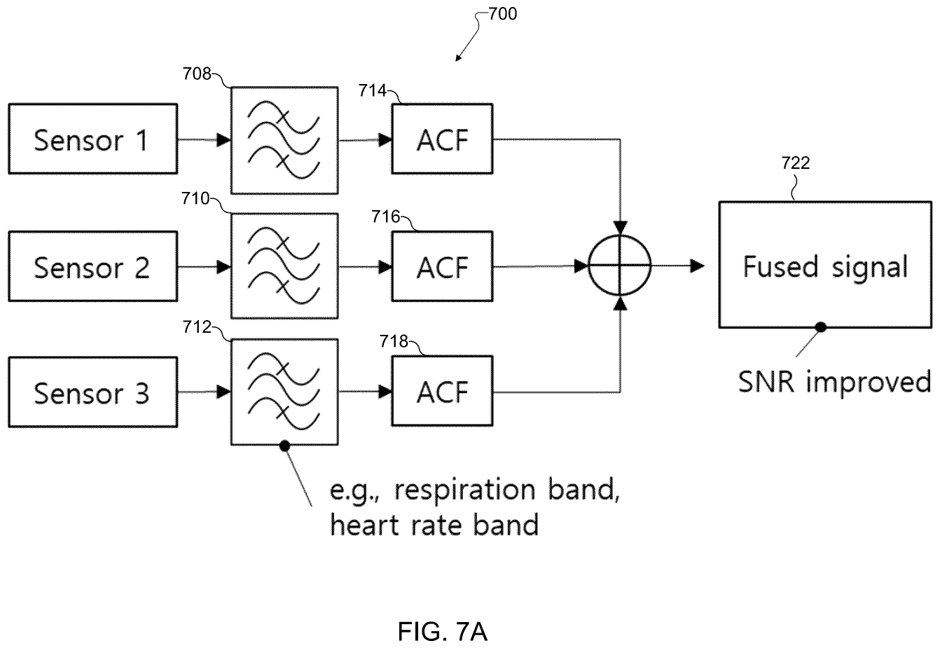

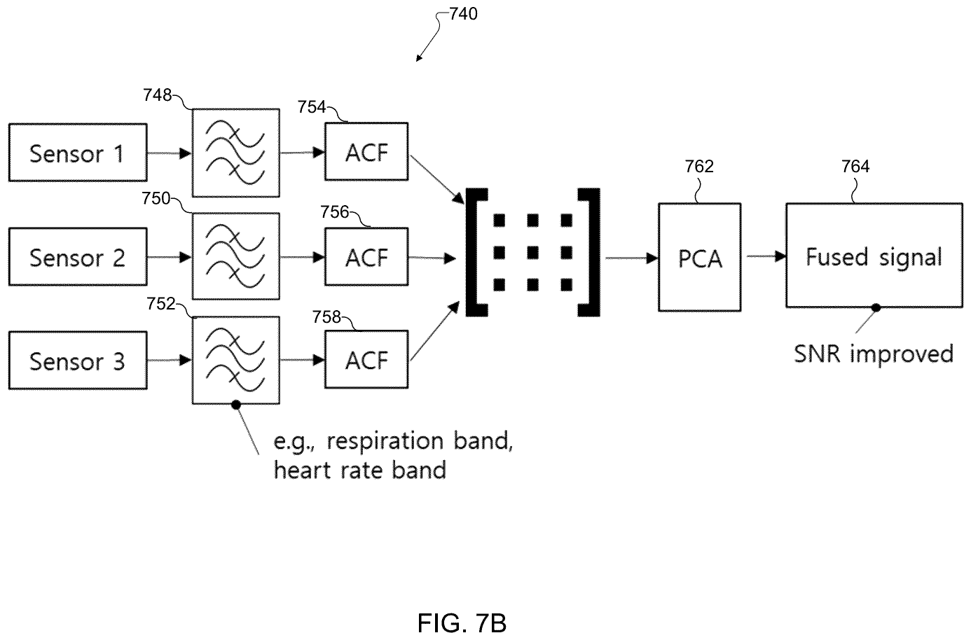

FIGS. 7A-7B illustrate example solutions for raw data level fusion 700 and 740 according to embodiments of the present disclosure. The example solutions for raw data level fusion 700 and 740 of FIGS. 7A-7B are for illustration only. Different embodiments of solutions for raw data level fusion 700 and 740 could be used without departing from the scope of this disclosure.

In one method of raw data level fusion, as illustrated in FIG. 7A, the raw data from each of sensor 1, sensor 2, and sensor 3 is filtered through bandpass filters 708, 710, and 712. After filtering, the data is then processed by auto correlation functions (ACFs) 714, 716, and 718. The ACFs process the data to imply the periodic property of the signals. Finally, the ACF processed data from different sensors is summed together to generate the fused signal 722, which has an improved SNR over the raw data.

In yet another method of raw data level fusion, as shown in FIG. 7B, the raw data from each of sensor 1, sensor 2, and sensor 3 is filtered through bandpass filters 758, 750, and 752. After filtering, the data is then processed by auto correlation functions (ACFs) 754, 756, and 758. The ACFs process the data to imply the periodic property of the signals. Finally principal component analysis (PCA) 762 is utilized to generate the fused signal 764, which has an improved SNR over the raw data.

Although FIGS. 7A-7B illustrate example solutions for raw data level fusion 700 and 740, various changes may be made to FIGS. 7A-7B. For example, the solutions for raw data level fusion 700 and 740 could include any number of each component described with respect to FIGS. 7A-7B. Also, various components described with respect to FIGS. 7A-7B could be combined, further subdivided, located in alternative locations, or omitted and additional components could be added according to particular needs.

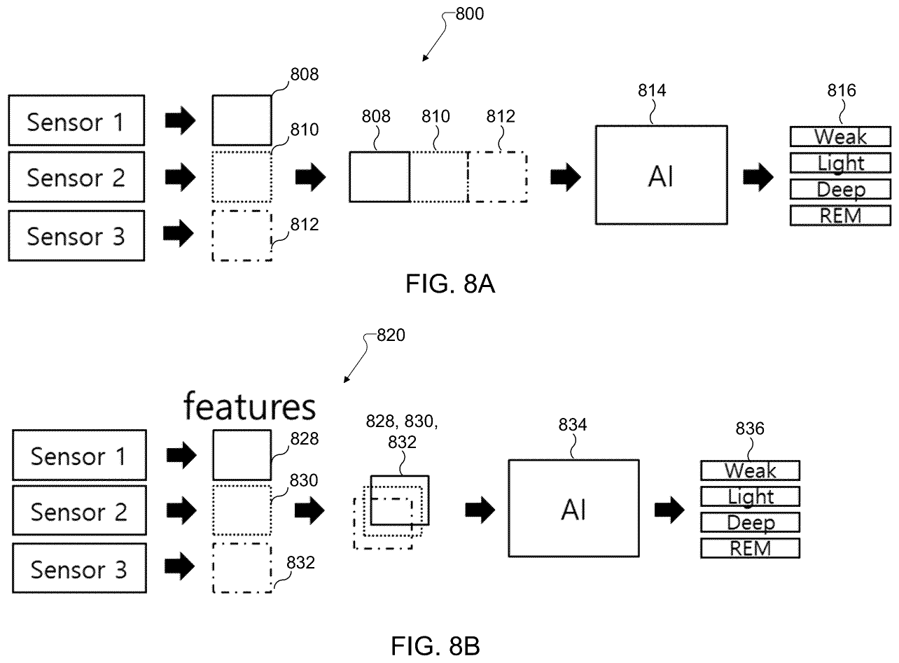

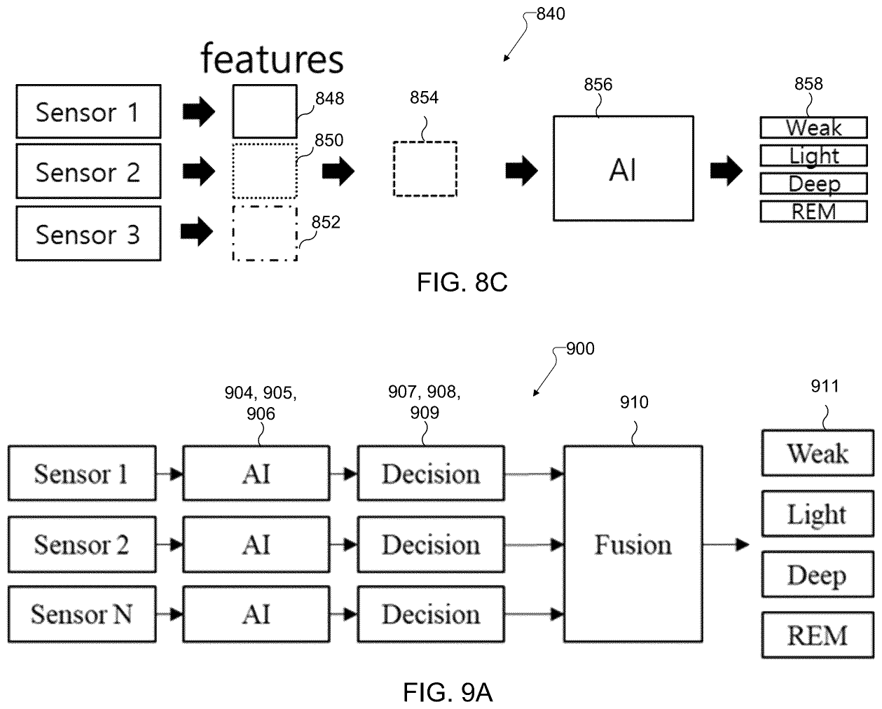

FIGS. 8A-8C illustrate example solutions for feature level fusion 800, 820, and 840 according to embodiments of the present disclosure. The example solutions for feature level fusion 800, 820, and 840 of FIGS. 8A-8C are for illustration only. Different embodiments of solutions for feature level fusion 800, 820, and 840 could be used without departing from the scope of this disclosure.

In one method of feature level fusion, multi-modality learning is used to boost the sleep stage decision performance. One example of multi-modality learning is illustrated in FIG. 8A, where sensor features are concatenated together and then input to the AI model. In the example of FIG. 8A, feature 808 from sensor 1, feature 810 from sensor 2, and feature 812 from sensor 3 are concatenated. The concatenated features are then processed by AI model 814 to determine a decision 816 (e.g., a sleep stage).

In another feature level fusion as illustrated in FIG. 8B, each sensor input is treated as a different channel for the AI input. The features included here could be a spectrogram or vital signs or statistics of vital signs. In the example of FIG. 8B, feature 828 from sensor 1, feature 830 from sensor 2, and feature 822 from sensor 3 are treated as different channels by AI 834. The features are then processed by AI model 834 to determine a decision 836 (e.g., a sleep stage).

In another method of feature level fusion as illustrated in FIG. 8C, the features from each sensor may first be processed and combined before being processed by the AI. For example, the features could be averaged, or the max or min or median value could be processed to get each corresponding value in the features among different sensors. In the example of FIG. 8C, feature 848 from sensor 1, feature 850 from sensor 2, and feature 852 from sensor 3 are processed and combined (e.g., averaged) to generate processed and combined features 854. The processed and combined features 854 are then processed by AI model 856 to determine a decision 858 (e.g., a sleep stage).

Although FIGS. 8A-8C illustrate example solutions for feature level fusion 800, 820, and 840, various changes may be made to FIGS. 7A-7B. For example, the solutions for feature level fusion 800, 820, and 840 could include any number of each component described with respect to FIGS. 8A-8C. Also, various components described with respect to FIGS. 8A-8C could be combined, further subdivided, located in alternative locations, or omitted and additional components could be added according to particular needs.

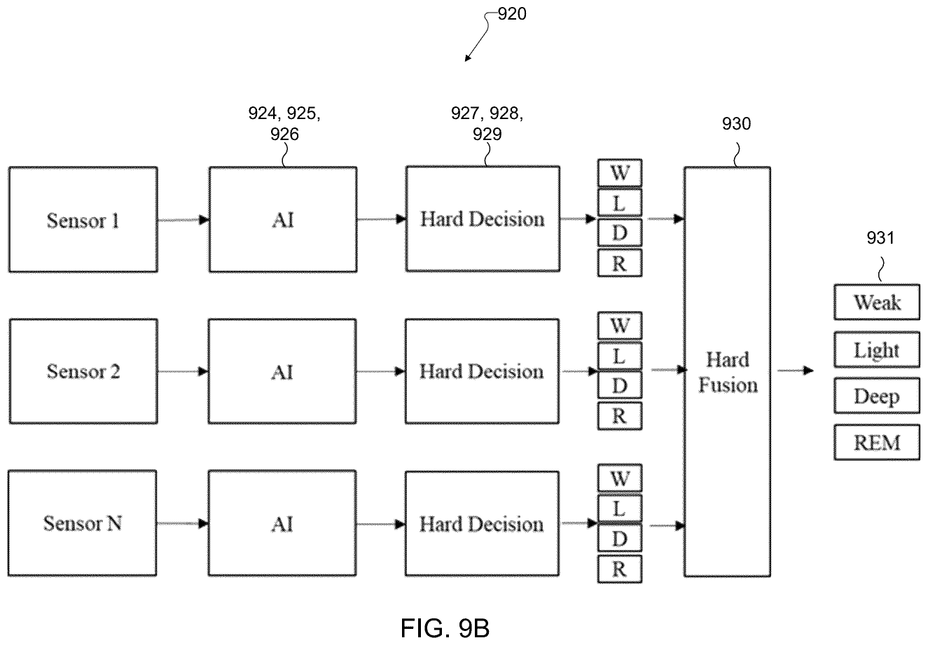

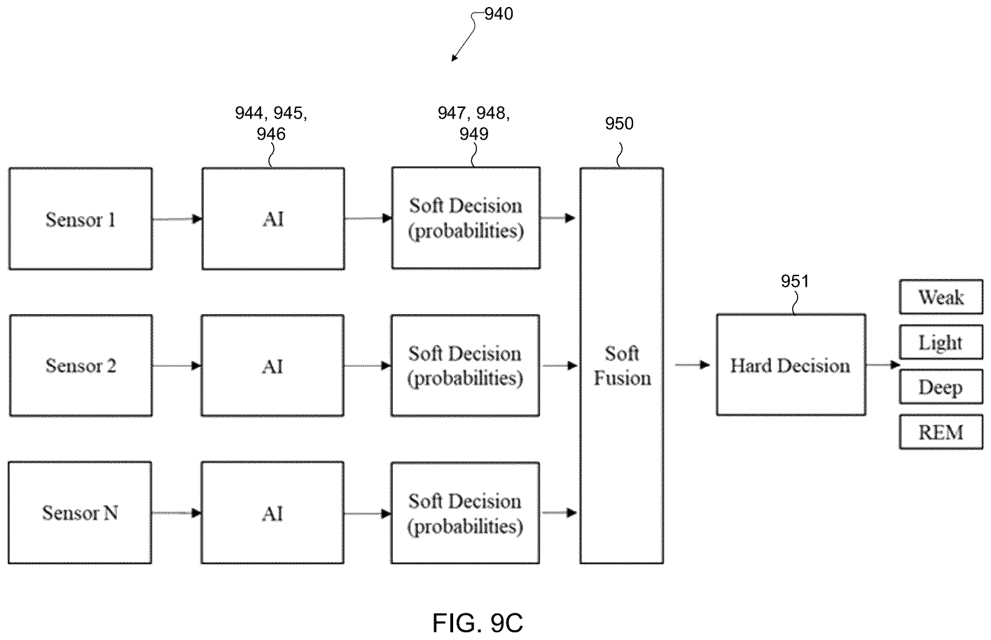

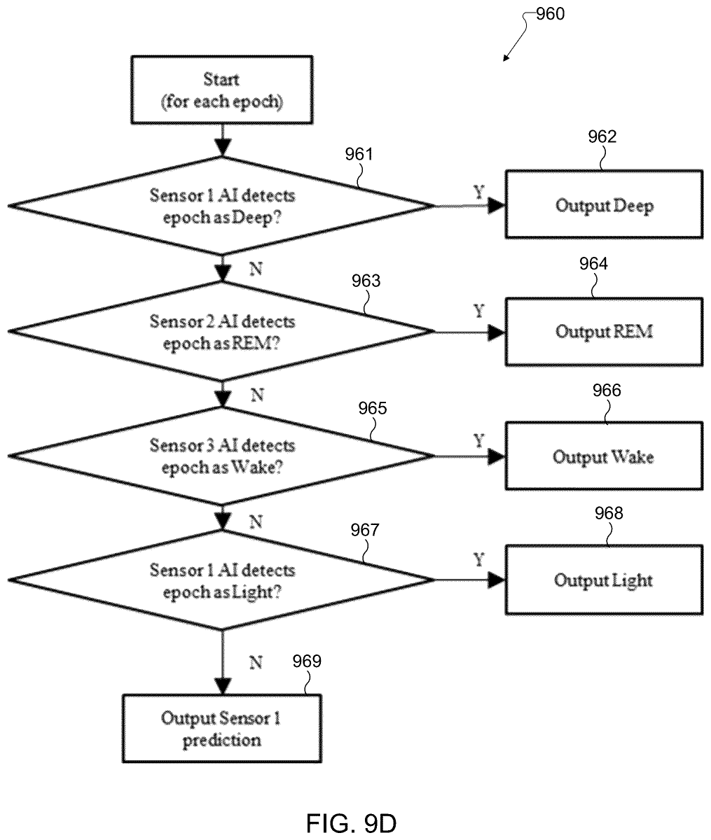

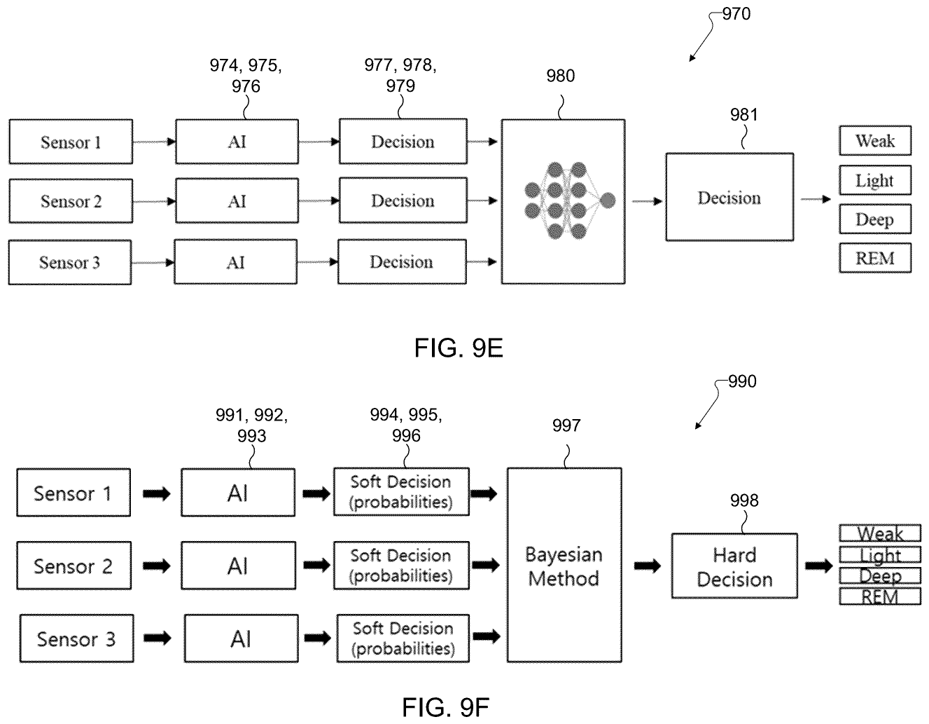

FIGS. 9A-9F illustrate example solutions for decision level fusion 900, 920, 940, 960, 970 and 990 according to embodiments of the present disclosure. The example solutions for decision level fusion 900, 920, 940, 960, 970 and 990 of FIGS. 9A-9F are for illustration only. Different embodiments of solutions for decision level fusion 900, 920, 940, 960, 970 and 990 could be used without departing from the scope of this disclosure.

In one method of decision level fusion, multi-model sensor decision fusion is used to boost the sleep stage decision performance. An example workflow of the solution is illustrated in FIG. 9A. In the example of FIG. 9A, AI-based sleep stage decisions 907, 908, and 909 corresponding to sensor 1, sensor 2, and sensor N (e.g., sensor 1 could be UWB, sensor 2 could be mmWave, sensor N could be Piezo, etc.) are generated separately by AI models 904, 905, and 906. Then those decisions are utilized as the input of a fusion algorithm 910 to generate the final sleep stage decision 911. Note the AI-based decisions can be either hard decisions (e.g., one of the four sleep stages) or soft decisions (e.g., probability vectors), correspondingly, the fusion algorithm 910 can be either a hard-fusion algorithm or a soft fusion algorithm, which are illustrated in FIG. 9B and FIG. 9C, respectively.

In the example of FIG. 9B, AI-based hard sleep stage decisions 927, 928, and 929 corresponding to sensor 1, sensor 2, and sensor N (e.g., sensor 1 could be UWB, sensor 2 could be mmWave, sensor N could be Piezo, etc.) are generated separately by AI models 924, 925, and 926. Then those decisions are utilized as the input of a hard fusion algorithm 930 to generate the final sleep stage decision 931.

In the example of FIG. 9C, AI-based soft sleep stage decisions 947, 948, and 949 corresponding to sensor 1, sensor 2, and sensor N (e.g., sensor 1 could be UWB, sensor 2 could be mmWave, sensor N could be Piezo, etc.) are generated separately by AI models 944, 945, and 946. Then those decisions are utilized as the input of a soft fusion algorithm 950 to generate the final hard sleep stage decision 951.

Regarding the hard-fusion algorithm, one embodiment utilizes different sensors to perform different sleep stage predictions. An example algorithm for such an embodiment is illustrated in FIG. 9D.

FIG. 9D illustrates a hard-fusion algorithm 960 utilizing different sensors according to embodiments of the present disclosure. An embodiment of the hard-fusion algorithm 960 illustrated in FIG. 9D is for illustration only. One or more of the components illustrated in FIG. 9D may be implemented in specialized circuitry configured to perform the noted functions or one or more of the components may be implemented by one or more processors executing instructions to perform the noted functions. Other embodiments of a hard-fusion algorithm 960 utilizing different sensors may be used without departing from the scope of this disclosure.

As illustrated in FIG. 9D, the hard-fusion algorithm 960 begins at step 961. At step 961, an AI model associated with Sensor 1 determines if the sleep epoch is deep. If sensor 1 detects the sleep epoch is Deep, the AI model outputs Deep at step 962. Otherwise, at step 963, an AI model associated with Sensor 2 determines if the sleep epoch is REM. If sensor 2 detects the sleep epoch is REM, the AI model outputs REM at step 964. Otherwise, at step 965, an AI model associated with Sensor 3 determines if the sleep epoch is Wake. If sensor 3 detects the sleep epoch is Wake, the AI model outputs Wake at step 966. Otherwise, at step 967, an AI model associated with Sensor 1 determines if the sleep epoch is Light. If sensor 1 detects the sleep epoch is Light, the AI model outputs Light at step 968. Otherwise, at step 969, the AI model outputs the prediction from sensor 1. For this type of algorithm, the choice of sensors, as well as the sequence of checking sleep stages, are the key factors that determine the final performance.

Although FIG. 9D illustrates one example of a hard-fusion algorithm 960 utilizing different sensors, various changes may be made to FIG. 9D. For example, while shown as a series of steps, various steps in FIG. 9D could overlap, occur in parallel, occur in a different order, or occur any number of times.

Regarding the soft-fusion algorithm, how to combine soft probabilities from all sensors is a key factor that determines the final performance. In one embodiment, averaging over probabilities may be utilized. This may be referred to as a mean fusion. For example, denote the soft probability vector from sensor n as:

Pₙ=[Pₙ^{W},Pₙ^{L},Pₙ^{D},Pₙ^{R}],

where Pₙ^{W}, Pₙ^{L}, Pₙ^{D}, and Pₙ^{R} represents the probability of sleep stage Wake, Light, Deep, and REM, respectively. Then the fusion algorithm output is:

$${P}_{fusion} = \frac{1}{N} {\sum }_{n = 1}^{N} {P}_{n} .$$

In one embodiment, a most confidence decision is chosen. A most confidence decision may refer to a decision with the highest confidence level, i.e., the sleep stage with the highest probability among all sensors is chosen:

Decision=argmax([P₁,P₂, . . . ,P_{N}]).

Additionally, a neural network can be utilized to train a learning-based soft-fusion algorithm, where soft decisions or hard decisions from each sensor's AI model are input to another neural network. This may be referred to as a learning-based fusion. The output will be the final sleep stage as shown in FIG. 9E.

In the example of FIG. 9E, AI-based sleep stage decisions 977, 978, and 979 corresponding to sensor 1, sensor 2, and sensor 3 (e.g., sensor 1 could be UWB, sensor 2 could be mmWave, sensor 3 could be Piezo, etc.) are generated separately by AI models 974, 975, and 976. Then those decisions are utilized as the input of a neural network 980 to generate the final sleep stage decision 981.

In yet another method, a Bayesian method is used to fuse different sensors sleep detection results. As illustrated in FIG. 9F, AI-based soft sleep stage decisions 994, 995, and 996 corresponding to sensor 1, sensor 2, and sensor 3 (e.g., sensor 1 could be UWB, sensor 2 could be mmWave, sensor 3 could be Piezo, etc.) are generated separately by AI models 944, 945, and 946. Each sensor outputs sleep stage probabilities based on its individual observation, then those probabilities are utilized to calculate the posterior probability 997 (i.e., a Bayesian method) based on all sensors' observations. To be specific, denote

p(S|Oₙ), S∈{Wake,Light,Deep,REM}

as the probability of sleep stage S, given sensor n's observation Oₙ. Then according to Bayes' theorem, the posterior probability of sleep stage S given on all sensors' observations can be written as

$$p ( S \vert {O}_{1} , {O}_{2} , ... , {O}_{n} ) = \frac{p ( {O}_{1} , {O}_{2} , ... {O}_{n} \vert S ) P ( S )}{p ( {O}_{1} , {O}_{2} , ... , {O}_{n} )} .$$

Under the assumption that each sensor's prediction is independent, and applying Bayes' theorem a second time, there can be

$$p ( S \vert {O}_{1} , {O}_{2} , ... , {O}_{n} ) ∝ ( {\prod }_{n = 1}^{N} p ( S \vert {O}_{n} ) ) {p ( S )}^{- ( N - 1 )} ,$$

here p(S|Oₙ) are from sensor outputs, and the prior probability of sleep stage p(S) can be obtained from an open sleep dataset available online. The final sleep stage decision 998 is obtained by:

$$Decision = arg {max}_{S} ( p ( S \vert {O}_{1} , {O}_{2} , ... , {O}_{n} ) ) .$$

Although FIGS. 9A-9F illustrate example solutions for decision level fusion 900, 920, 940, 960, 970 and 990, various changes may be made to FIGS. 9A-9F. For example, the solutions for decision level fusion 900, 920, 940, 960, 970 and 990 could include any number of each component described with respect to FIGS. 9A-9F. Also, various components described with respect to FIGS. 9A-9F could be combined, further subdivided, located in alternative locations, or omitted and additional components could be added according to particular needs.

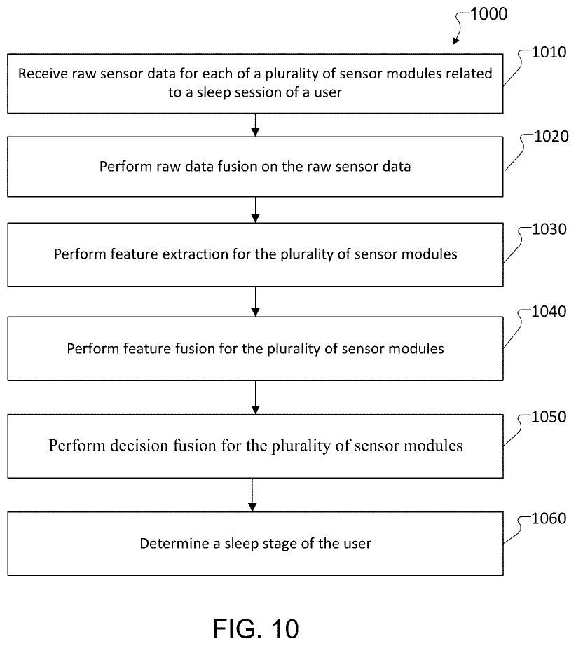

FIG. 10 illustrates a method 1000 for sleep detection with multiple sensors according to embodiments of the present disclosure. An embodiment of the method illustrated in FIG. 10 is for illustration only. One or more of the components illustrated in FIG. 10 may be implemented in specialized circuitry configured to perform the noted functions or one or more of the components may be implemented by one or more processors executing instructions to perform the noted functions. Other embodiments of a method 1000 for sleep detection with multiple sensors may be used without departing from the scope of this disclosure.

As illustrated in FIG. 10, the method 1000 begins at step 1010. At step 1010, a sleep monitoring apparatus receives raw sensor data related to a sleep session of a user of the sleep monitoring apparatus from each of a plurality of sensor modules. At step 1020, the sleep monitoring apparatus performs raw data fusion on the raw sensor data. The raw data fusion generates a fused raw data signal. At step 1030, the sleep monitoring apparatus performs feature extraction for the plurality of sensors. The feature extraction may be based on the fused raw data signal. At step 1040, the sleep monitoring apparatus performs feature fusion for the plurality of sensor modules. The feature fusion may be based on the feature extraction. At step 1050, the sleep monitoring apparatus performs decision fusion for the plurality of sensor modules. The decision fusion may be based on the feature fusion. Finally, at step 1060, the sleep monitoring apparatus determines a sleep stage of the user. The determination of the sleep stage may be based on any of the raw data fusion, the feature fusion, and the decision fusion.

Although FIG. 10 illustrates one example of a method 1000 for sleep detection with multiple sensors, various changes may be made to FIG. 10. For example, while shown as a series of steps, various steps in FIG. 10 could overlap, occur in parallel, occur in a different order, or occur any number of times.

Any of the above variation embodiments can be utilized independently or in combination with at least one other variation embodiment. The above flowcharts illustrate example methods that can be implemented in accordance with the principles of the present disclosure and various changes could be made to the methods illustrated in the flowcharts herein. For example, while shown as a series of steps, various steps in each figure could overlap, occur in parallel, occur in a different order, or occur multiple times. In another example, steps may be omitted or replaced by other steps.

Although the present disclosure has been described with exemplary embodiments, various changes and modifications may be suggested to one skilled in the art. It is intended that the present disclosure encompass such changes and modifications as fall within the scope of the appended claims. None of the description in this application should be read as implying that any particular element, step, or function is an essential element that must be included in the claim scope. The scope of patented subject matter is defined by the claims.

What is claimed is:

1. A sleep monitoring apparatus comprising:
a plurality of sensor modules;
a transceiver; and
a processor operatively coupled with the plurality of sensor modules and the transceiver, the processor configured to:
receive, from the plurality of sensor modules, raw sensor data for each of the plurality of sensor modules related to a sleep session of a user of the sleep monitoring apparatus;
perform raw data fusion of the raw sensor data, wherein the raw data fusion generates a fused raw data signal;
based on the fused raw data signal, perform feature extraction for the plurality of sensor modules;
based on the feature extraction, perform feature fusion for the plurality of sensor modules;
based on the feature fusion, perform decision fusion for the plurality of sensor modules; and
determine a sleep stage of the user based on the decision fusion.

2. The sleep monitoring apparatus of claim 1, wherein to perform the raw data fusion on the raw sensor data, the processor is further configured to:
filter the raw sensor data from each sensor module;
perform an auto correlation function (ACF) on the filtered raw sensor data from each sensor module, wherein the ACF generates ACF processed sensor data; and
generate the fused raw data signal based on the ACF processed sensor data.

3. The sleep monitoring apparatus of claim 2, wherein to generate the fused raw data signal based on the ACF, the processor is further configured to:
sum the ACF processed sensor data; or
perform a principal component analysis (PCA) on the ACF processed sensor data.

4. The sleep monitoring apparatus of claim 1, wherein to perform the feature fusion, the processor is further configured to:
determine a feature for each sensor module of the plurality of sensor modules;
fuse the features, wherein to fuse the features, the processor is further configured to:
concatenate the features;
treat each feature as a different channel; or
process the features; and
based on the fused features, determine a sleep stage of the user.

5. The sleep monitoring apparatus of claim 1, wherein to perform the decision fusion, the processor is further configured to:
determine hard sleep stage decision for each sensor module of the plurality of sensor modules;
perform a hard fusion of the hard sleep stage decisions; and
generate a final sleep stage decision based on the hard fusion.

6. The sleep monitoring apparatus of claim 1, wherein to perform the decision fusion, the processor is further configured to:
determine a soft sleep stage decision for each sensor module of the plurality of sensor modules;
perform a soft fusion of the soft sleep stage decisions;
determine a hard decision based on the soft fusion; and
generate a sleep stage decision based on the hard decision.

7. The sleep monitoring apparatus of claim 6, wherein to perform the soft fusion, the processor is further configured to perform at least one of:
a mean fusion;
a most confidence decision;
a Bayesian method-based fusion; and
a learning-based fusion.

8. A method of operating a sleep monitoring apparatus, the method comprising:
receiving, from a plurality of sensor modules, raw sensor data for each of the plurality of sensor modules related to a sleep session of a user of the sleep monitoring apparatus;
performing raw data fusion of the raw sensor data, wherein the raw data fusion generates a fused raw data signal;
based on the fused raw data signal, performing feature extraction for the plurality of sensor modules;
based on the feature extraction, performing feature fusion for the plurality of sensor modules;
based on the feature fusion, performing decision fusion for the plurality of sensor modules; and
determining a sleep stage of the user based on the decision fusion.

9. The method of claim 8, wherein performing the raw data fusion on the raw sensor data comprises:
filtering the raw sensor data from each sensor module;
performing an auto correlation function (ACF) on the filtered raw sensor data from each sensor module, wherein the ACF generates ACF processed sensor data; and
generating the fused raw data signal based on the ACF processed sensor data.

10. The method of claim 9, wherein generating the fused raw data signal based on the ACF comprises:
summing the ACF processed sensor data; or
performing a principal component analysis (PCA) on the ACF processed sensor data.

11. The method of claim 8, wherein performing the feature fusion comprises:
determining a feature for each sensor module of the plurality of sensor modules;
fusing the features, wherein fusing the features comprises:
concatenating the features;
treating each feature as a different channel; or
processing the features; and
based on the fused features, determining a sleep stage of the user.

12. The method of claim 8, wherein performing the decision fusion comprises:
determining hard sleep stage decision for each sensor module of the plurality of sensor modules;
performing a hard fusion of the hard sleep stage decisions; and
generating a final sleep stage decision based on the hard fusion.

13. The method of claim 8, wherein performing the decision fusion comprises:
determining a soft sleep stage decision for each sensor module of the plurality of sensor modules;
performing a soft fusion of the soft sleep stage decisions;
determining a hard decision based on the soft fusion; and
generating a sleep stage decision based on the hard decision.

14. The method of claim 13, wherein performing the soft fusion comprises performing at least one of:
a mean fusion;
a most confidence decision;
a Bayesian method-based fusion; and
a learning-based fusion.

15. A non-transitory computer readable medium embodying a computer program, the computer program comprising program code that, when executed by a processor of a device, causes the device to:
receive, from a plurality of sensor modules, raw sensor data for each of the plurality of sensor modules related to a sleep session of a user of the sleep monitoring apparatus;
perform raw data fusion of the raw sensor data, wherein the raw data fusion generates a fused raw data signal;
based on the fused raw data signal, perform feature extraction for the plurality of sensor modules;
based on the feature extraction, perform feature fusion for the plurality of sensor modules;
based on the feature fusion, perform decision fusion for the plurality of sensor modules; and
determine a sleep stage of the user based on the decision fusion.

16. The non-transitory computer readable medium of claim 15, wherein to perform the raw data fusion on the raw sensor data, the computer program further comprises program code that, when executed by the processor of the device causes the device to:
filter the raw sensor data from each sensor module;
perform an auto correlation function (ACF) on the filtered raw sensor data from each sensor module, wherein the ACF generates ACF processed sensor data; and
generate the fused raw data signal based on the ACF processed sensor data.

17. The non-transitory computer readable medium of claim 16, wherein to generate the fused raw data signal based on the ACF, the computer program further comprises program code that, when executed by the processor of the device causes the device to:
sum the ACF processed sensor data; or
perform a principal component analysis (PCA) on the ACF processed sensor data.

18. The non-transitory computer readable medium of claim 15, wherein to perform the feature fusion, the computer program further comprises program code that, when executed by the processor of the device causes the device to:
determine feature for each sensor module of the plurality of sensor modules;
fuse the features, wherein to fuse the features the computer program further comprises program code that, when executed by the processor of the device causes the device to:
concatenate the features;
treat each feature as a different channel; or
process the features; and
based on the fused features, determine a sleep stage of the user.

19. The non-transitory computer readable medium of claim 15, wherein to perform the decision fusion, the computer program further comprises program code that, when executed by the processor of the device causes the device to:
determine a hard sleep stage decision for each sensor module of the plurality of sensor modules;
perform a hard fusion of the hard sleep stage decisions; and
generate a final sleep stage decision based on the hard fusion.

20. The non-transitory computer readable medium of claim 15, wherein to perform the decision fusion, the computer program further comprises program code that, when executed by the processor of the device causes the device to:
determine a soft sleep stage decision for each sensor module of the plurality of sensor modules;
perform a soft fusion of the soft sleep stage decisions, wherein to perform the soft fusion the computer program further comprises program code that, when executed by the processor of the device causes the device to perform at least one of:
a mean fusion,
a most confidence decision,
a Bayesian method-based fusion, and
a learning-based fusion;
determine a hard decision based on the soft fusion; and
generate a sleep stage decision based on the hard decision.
